(19) United States
(12) Patent Application Publication
(10) Pub. No.: US 2017/0360502 A1
(43) Pub. Date: Dec. 21, 2017

(54) ELECTRODE CATHETER DEVICE

(71) Applicant: Peter Osypka, Grenzach-Wyhlen (DE)

(72) Inventor: Peter Osypka, Grenzach-Wyhlen (DE)

(21) Appl. No.: 15/225,869

(22) Filed: Aug. 2, 2016

Related U.S. Application Data
(63) Continuation-in-part of application No. 13/908,834, filed on Jun. 3, 2013, now abandoned.

(30) Foreign Application Priority Data
Jun. 6, 2012 (EP) 12004329

(51) Int. Cl. A61B 18/14; A61N 1/05; A61B 18/00

(52) U.S. Cl. CPC A61B 18/1492; A61N 1/0573; A61B 2018/00577; A61B 2018/00351; A61B 2018/00636

(57) ABSTRACT

An implantable electrode catheter device comprising an inner electrode catheter and an outer electrode catheter. The outer electrode catheter including a catheter shaft having at least one electrode at a distal end and a lumen to receive the inner electrode catheter therein. The outer electrode is adjustable or movable relative to the inner electrode catheter in an axial direction. The inner electrode catheter has a fixation element disposed at a distal end. The inner electrode catheter together with the fixation element forms an indifferent electrode whereby radio frequency catheter ablation occurs between the electrode and the indifferent electrode.

### CROSS-REFERENCE TO RELATED APPLICATION

The subject application is a continuation-in-part of U.S. application Ser. No. 13/908,834, filed Jun. 3, 2013, the disclosure of which is herein incorporated by reference in its entirety.

### BACKGROUND OF THE INVENTION

### Field of the Invention

The present invention relates to an ablation device and more particularly for the ablation of tissue in a body cavity of a human or an animal, especially used to treat atrial fibrillation.

### Description of Related Art

Atrial fibrillation is a frequent finding especially in elderly patients and occurs when the normal electrical impulses that are generated by the SA node are overwhelmed by disorganized electrical impulses in the atria. These disorganized impulses cause the muscles of the upper chamber of the heart to fibrillate and this leads to the conduction of irregular impulses to the ventricles and may lead to acute hemodynamic instability.

In the treatment of atrial fibrillation, for example, a radio frequency (RF) ablation catheter equipped with a number of electrodes can be brought into contact with cardiac tissue according to the so called “Maze procedure” for creating one or more ablation points or an ablation path along the tissue especially around the pulmonary veins. To create an ablation path the catheter must have a large number of electrodes and can thus only be produced at high costs. Furthermore, the exact guidance of the catheter is difficult and time consuming As the guidance is done under x-ray control the exposure to x-rays is high.

U.S. Patent Application Publication No. 2009/0312755 discloses an ablation device comprising an elongate shaft and a positioning mechanism and an energy delivery element adjacent the distal end of the shaft said energy delivery element is adapted to create a zone of ablation.

Such conventional methods and systems have generally been considered satisfactory for their intended purpose. However, there is still a need in the art for an electrode catheter device that allows improved treatment of atrial fibrillation by creating a specifically designed ablation path in various locations in the heart. The present invention provides a solution for these problems.

### SUMMARY OF THE INVENTION

The subject invention is directed to a new and useful electrode catheter device.

The present invention provides an implantable electrode catheter device comprising an inner electrode catheter and an outer electrode catheter. The outer electrode catheter includes a catheter shaft having at least one electrode pole located at its distal end. The outer electrode catheter is further provided with an insertion opening and thus with a lumen to receive the inner electrode catheter therein.

The inner electrode catheter is provided with a lumen, in which stylets (mandrins) in different forms can be inserted. There are mainly two types of stylets. One stylet has a flattened end. This stylet is used to screw in the tip of the inner electrode catheter into the tissue and to guide the whole device within the left atrium. The other stylet is curved and is used to create a specific ablation path.

The outer electrode is adjustable or movable relative to the inner electrode catheter in an axial direction. The inner electrode catheter has a fixation element disposed at a distal end. The inner electrode catheter together with the fixation element forms an indifferent electrode pole whereby radio frequency catheter ablation occurs between the electrode pole placed on the outer catheter and said indifferent electrode pole. A high frequency voltage is applied between the electrode pole placed on the outer catheter and the indifferent electrode pole thereby heating the subjacent tissue and creating an ablation path.

The fixation element is preferably a helical screw or a magnet and allows the fixation of the electrode at any place in the left atrium or if desired in the right atrium or the ventricles. The fixation of the screw is done by aid of the stylet. The stylet has a flattened tip fitting in a slit inside the screw tip of the inner electrode catheter and by turning the stylet enables the screw to turn clockwise or counterclockwise.

The fixation element could also be a magnet at the tip of the inner catheter, if within the esophagus close to the left atrium, another catheter with a magnet is positioned.

The movable outer catheter carries at least one electrode pole for energy absorption or energy release. The electrode pole is made of metal or of conductive plastic and is located at the inner wall of the left atrium or if desired the inner wall of the right atrium. The electrode pole of the outer electrode catheter may be designed in cylindrical shape or is in shape of an electrically conducting segment.

The ablation temperature may be controlled by a temperature sensor embedded proximate the distal end on the catheter shaft.

The shaft of the outer electrode catheter may include a red light emitting diode (LED) at its distal end thus helping to control the position of the electrode catheter device during the ablation process. Preferably the emitting diode (LED) is positioned in opposite position of the segment pole. This opposite position of the LED allows a better control of the position of the different electrode pole (segment pole).

The shaft of the outer electrode catheter may be made of flexible plastic.

The ablation process is not limited to the ablation of heart tissue. Further tissue such as for example kidney tissue or stomach tissue may also be ablated

A method for creating an ablation path is disclosed. The method comprises the following steps: with a steerable guiding catheter the ablation device catheter is guided trans-septal to the desired location within the left atrium of the heart. With the aid of the stylet inside the inner catheter, the tip is screwed into the tissue of the inner left atrium. Guiding catheter and stylet are withdrawn and depending of the expected ablation line the appropriate special formed stylet is introduced. The helical screw is electrically conducting with the coil of the inner electrode catheter and combined they form an indifferent electrode

An outer electrode catheter is guided over the inner electrode catheter such that the outer electrode catheter concentrically encases the inner electrode catheter. A shaft of the outer electrode catheter includes an electrode pole. A high frequency voltage is applied between the electrode pole of the outer electrode catheter and the indifferent electrode pole thereby heating subjacent tissue and creating an ablation path.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

So that those skilled in the art to which the subject invention appertains will readily understand how to make and use the devices and methods of the subject invention without undue experimentation, preferred embodiments thereof will be described in detail herein below with reference to certain figures, wherein:

FIG. 1 is a side view of the electrode catheter device according to the invention implanted in the left atrium of the heart;

FIG. 2A is a detailed view of the catheter device of FIG. 1, showing the different pole in a cylindrical shape;

FIG. 2B is a detailed view of the catheter device of FIG. 1, showing an electrically conducting segment shape;

FIG. 2C is a detailed view of the catheter device of FIG. 1, showing a magnet as a fixation device;

FIG. 2D is a detailed view of the catheter device of FIG. 1, showing a temperature sensor;

FIG. 3 shows different forms of the stylet;

FIG. 4 shows the positioning of the inventive electrode catheter device at the inner wall of the left atrium when retracting the catheter during radio frequency ablation;

FIG. 5 shows the positioning of the inventive electrode catheter device at the inner wall of the left atrium when retracting and rotating the catheter during radio frequency ablation;

FIG. 6 shows positioning of the electrode catheter fixed at the inner wall of the atrium wherein after retracting the ablation line will be formed accordingly;

FIG. 7 shows positioning of the electrode catheter fixed at the inner wall of the atrium wherein after retracting the ablation line will be formed accordingly; and

FIG. 8 shows positioning of the electrode catheter wherein an ablation path around the pulmonary veins is possible.

### DETAILED DESCRIPTION OF THE PREFERRED EMBODIMENTS

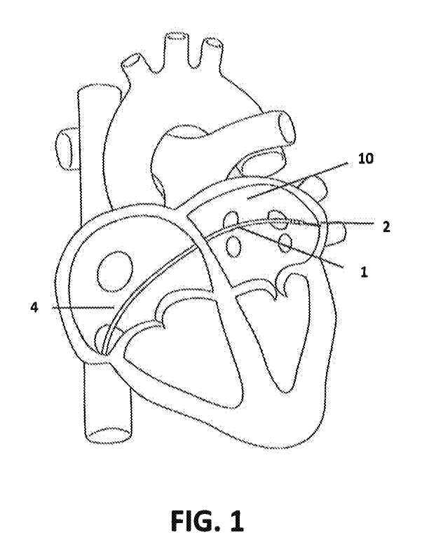

Reference will now be made to the drawings wherein like reference numerals identify similar structural features or aspects of the subject invention. For purposes of explanation and illustration, and not limitation, a partial view of an exemplary embodiment of the electrode catheter device in accordance with the invention is shown in FIG. 1 and is designated generally by reference character 1. The system of the invention can be used for the treatment of atrial fibrillation.

FIG. 1 shows an electrode catheter device 1 implanted in the left atrium 10 of the heart, which is anchored with a helical screw 2 at the inner wall of the left atrium. The location of the anchor may be anywhere at the tissue of the atrium. The access to the left atrium 10 may be achieved by trans-septal puncture from the right atrium through the intra-atrial septum using known methods.

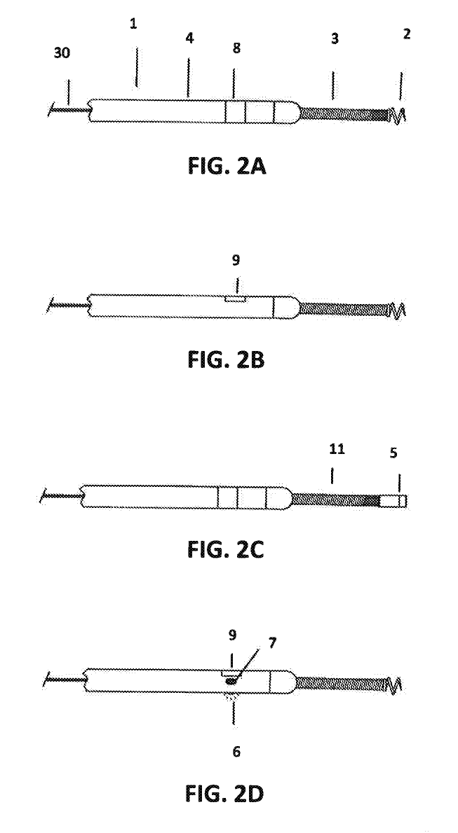

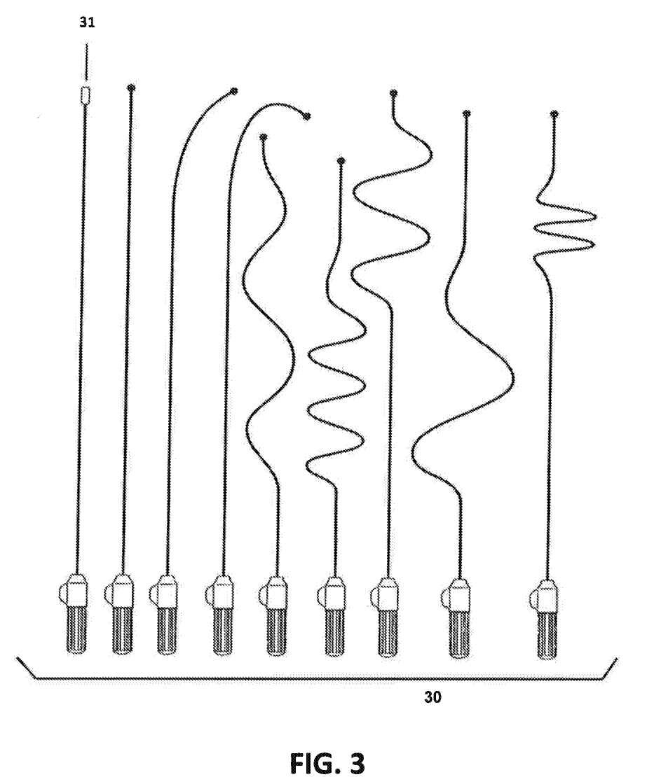

At first the inner electrode catheter 3 is guided to the desired target in the left atrium 10 by means of a steerable catheter device (not shown) When the distal end of the inner electrode catheter 3 has reached its target in the left atrium 10, the inner electrode catheter 3 is fixed at the inner wall of the left atrium 10 by means of a fixation device such as for example by means of a helical screw 2 (FIGS. 2a and 2b) or a magnet 5 (FIG. 2c). In case the fixation element is a helical screw 2, the stylet as shown in FIG. 3 having a flattened tip 31 is used to screw in said screw into the tissue. Then the steerable catheter and the stylet are retracted. Then the outer electrode catheter 4 is guided over the inner electrode catheter 3 so that the outer electrode catheter 4 concentrically encases the inner electrode catheter 3.

FIGS. 2A-2D show a detailed view of the inventive catheter devices 1 showing the inner electrode catheter 3 disposed within the outer electrode catheter 4. The shaft of the outer electrode catheter 4 carries the different pole 8 which is made from metal or from conductive plastic such as conductive polyurethane or silicone. The shape of the outer different pole is variable. The different pole 8 of the outer electrode catheter 4 may be designed in cylindrical shape (FIGS. 2A, 2C) or is in shape of an electrically conducting segment pole 9 (FIG. 2B, 2D). The inner electrode catheter is provided with a lumen, in which stylets (mandrins) 30 in different forms according to FIG. 3 can be inserted. The stylet according to FIG. 3 with the flattened tip 31 is used to screw in the tip of the inner electrode catheter into the tissue and to guide the whole device within the left atrium. The different forms of stylets as shown in FIG. 3 are inserted to draw the desired ablation path.

The inner electrode catheter 3 preferably comprises together with the screw an electrically conducting coil shaped electrode 11 or an electrically conducting strand or braid which is torsional rigid.

FIG. 2D shows a detailed view of the electrode catheter device 1 with the helical screw 2. The shaft of the outer electrode catheter 4 also includes a red light emitting diode (LED) 6 and/or a temperature sensor 7 embedded proximate the distal end or as shown in FIG. 2D the LED 6 is positioned in opposite position of the segment pole 9. The ablation temperature is controlled by receiving feedback from the temperature sensor.

The red light emitting diode 6 shows the position of the electrode catheter device 1 without submitting x-rays to the patient as done in common ablation processes due to an x-ray sensor placed e.g. in the esophagus or in a vein.

FIG. 3 shows some forms of stylets (mandrins). The left most stylet is used to fix the screw tip of the inner electrode at the inner wall of the atrium. The stylet has a flattened tip (31), fitting in a slit inside the screw tip of the inner electrode catheter and by turning the stylet enables the screw to turn clockwise or counterclockwise. Furthermore some additional forms of stylets are shown in FIG. 3. When inserted into the catheter, the stylet can be used to draw different ablation lines within the left atrium. Any shapes or curves in different plains are applicable as long as the outer catheter is gliding during retraction. The material of the stylets is stainless steel or preferably Nitinol. Especially remarkable is the stylet shown to the far right. When inserted into the catheter the stylet can be used to draw an ablation line around the pulmonary veins.

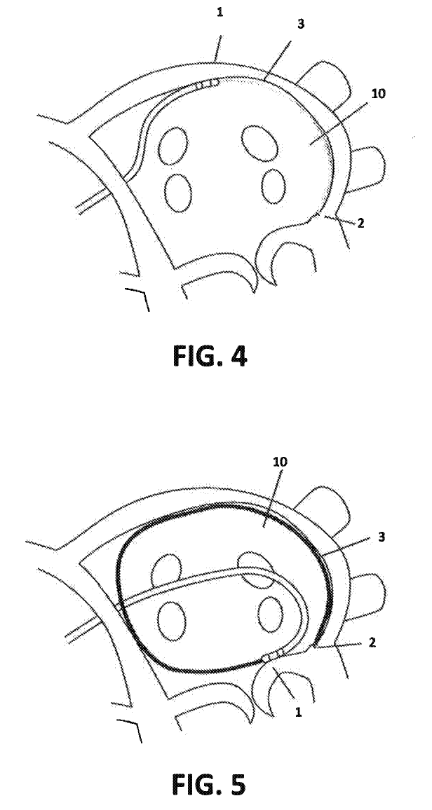

FIG. 4 shows the positioning of the inventive electrode catheter device 1 at the inner wall of the left atrium 10. Due to the inserted special formed stylet according to FIG. 3 the catheter device will follow the shape of the inserted stylet. When retracting the catheter during radio frequency ablation the ablation line will be formed accordingly. The electrically conductive coil of the inner electrode catheter 3 together with the helical screw 2 forms the indifferent electrode pole. Between the electrode 8 and the indifferent electrode high frequency voltage of about 500 kHz is applied thus heating the subjacent tissue and thus creating an ablation path. When retracting the outer electrode catheter 4 the surface of the indifferent electrode increases.

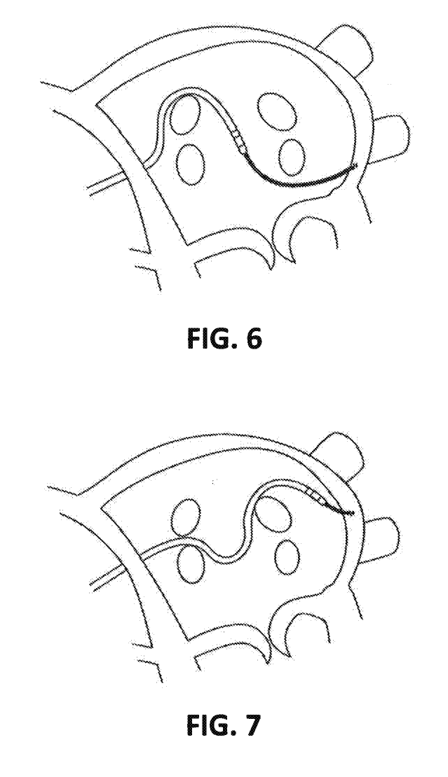

It is important that the inner electrode catheter 3 may be placed at any place in the left atrium 10. Depending on the position of the fixation point in the tissue of the left atrium, preferably in the inner wall and with the insertion of a formed stylet it is possible to draw the ablation path in the whole atrium and even around the pulmonary veins. Each and every part of the atrium can be reached. The possibility of reaching each and every part of the left atrium is further supported by being able to rotate the electrode catheter device 1 when retracting the device together with the stylet as shown in FIG. 5.

FIG. 5 shows the positioning of the inventive electrode catheter device 1 at the inner wall of the left atrium 10 when retracting the catheter during radio frequency ablation. By withdrawing the stylet to a certain position and rotating the outer catheter 4 for 180 degrees a change of position occurs as shown in FIG. 5. Depending on the position of fixation and on the position where the rotation occurs each part of the left atrium 10 can be reached to create an ablation path. Even the ablation around the pulmonary veins is possible.

FIG. 6 shows the positioning of the inventive electrode catheter device (1) fixed at the inner wall of the left atrium (10) comprising a special formed stylet according to FIG. 3. When retracting the outer catheter during radio frequency ablation, the ablation line will be formed accordingly.

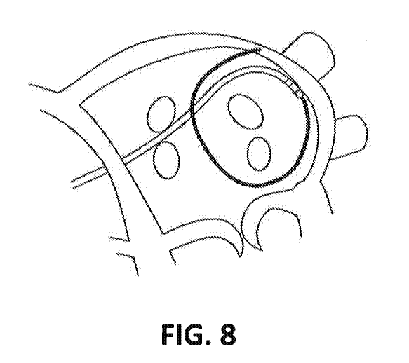

FIG. 7 shows the positioning of the inventive electrode catheter device (1) fixed at the inner wall of the left atrium (10) comprising a special formed stylet according to FIG. 3. When retracting the outer catheter during radio frequency ablation, the ablation line will be formed accordingly

FIG. 8 Depending on the position of fixation and on the position where the rotation occurs, each part of the left atrium can be reached to create an ablation path. Even the ablation around the pulmonary veins is possible.

If desired, the present ablation process can be combined with a chemical ablation process using sodium chloride. The device described hereinabove is referenced treating heart tissue, however it is understood that the ablation process is not limited to the ablation of heart tissue. Further tissue such as for example kidney tissue or stomach tissue may also be ablated.

The methods and systems of the present invention, as described above and shown in the drawings, provide for an electrode catheter device with superior properties. While the apparatus and methods of the subject invention have been shown and described with reference to preferred embodiments, those skilled in the art will readily appreciate that changes and/or modifications may be made thereto without departing from the spirit and scope of the subject invention.

1. An implantable electrode catheter device comprising:
an inner electrode catheter; and
an outer electrode catheter, the outer electrode catheter comprising:
a catheter shaft having at least one electrode pole located at a distal end; and
a lumen which can receive the inner electrode catheter therein, the outer electrode catheter is adjustable or movable relative to the inner electrode catheter in an axial direction, wherein the inner electrode catheter is provided with a stylet and has a fixation element disposed at the distal end, the inner electrode catheter together with the fixation element forms an indifferent electrode pole whereby radio frequency catheter ablation occurs between the electrode pole of the outer electrode catheter and the indifferent electrode pole.

2. The electrode catheter device according to claim 1, wherein the fixation element of the inner electrode catheter is a helical screw.

3. The electrode catheter device according to claim 1, wherein the outer electrode catheter is provided at its distal end with a segmented electrode pol and a red light emitting diode (LED) at the opposite site of the segment pol and/or a temperature sensor placed under the segment pol.

4. The electrode catheter device according to claim 1, wherein the outer electrode catheter includes a red light emitting diode (LED) embedded proximate a distal end.

5. The electrode catheter device according to claim 1, wherein the outer electrode catheter carries at least one electrode pole for energy absorption.

6. The electrode catheter device according to claim 1, wherein the electrode pole of the outer electrode catheter is made of metal.

7. The electrode catheter device according to claim 1, wherein the electrode pole of the outer electrode catheter is designed in cylindrical shape.

8. The electrode catheter device according to claim 2, wherein the inner electrode catheter comprises an electrically conducting coil shaped electrode pole or an electrically conducting strand or braid such that the-coil shaped electrode pole or the conducting strand or braid together with the helical screw form the indifferent electrode.

9. The electrode catheter device according to claim 1, wherein the shaft of the outer electrode catheter is made of flexible plastic.

10. The electrode catheter device according to claim 1, wherein high frequency voltage is applied between the electrode pole of the outer electrode catheter and the indifferent electrode pole thereby heating the subjacent tissue and creating an ablation path.

11. The electrode catheter device according to claim 1, wherein the fixation element of the inner electrode catheter is a magnet.

12. The electrode catheter device according to claim 1, wherein the outer electrode catheter includes a temperature sensor embedded proximate a distal end.

13. The electrode catheter device according to claim 1, wherein the outer electrode catheter carries at least one electrode for energy release.

14. The electrode catheter device according to claim 1, wherein the electrode of the outer electrode catheter is made of conductive plastic.

15. The electrode catheter device according to claim 1, wherein the electrode of the outer electrode catheter is designed in the shape of an electrically conducting segment.

16. A method for treatment of creating an ablation path, the method comprising:
guiding an inner electrode catheter trans-septal by means of a steerable guiding catheter to a desired target in the left atrium of a patient, wherein the inner electrode catheter has an electrically conducting coil;
fixing the distal end of the inner electrode catheter at the inner wall of the left atrium with a helical screw by the aid of a stylet having a flattened tip or with a magnet such that the electrically conducting coil of the inner electrode catheter and the helical screw form an indifferent electrode pole;
retracting the guiding catheter and the stylet having a flattened tip,
guiding an outer electrode catheter over the inner electrode catheter such that the outer electrode catheter concentrically encases the inner electrode catheter, wherein a shaft of the outer electrode catheter includes an electrode pole;
inserting the appropriate special formed stylet; and
applying a high frequency voltage between the electrode pole of the outer electrode catheter and the indifferent electrode pole thereby heating subjacent tissue and creating an ablation path.
